(19) United States
(12) Patent Application Publication
(10) Pub. No.: US 2003/0004515 A1
(43) Pub. Date: Jan. 2, 2003

(54) Resorbable bone plate

(76) Inventors: Raymond Curtis, Davos (CH); Markus Hehli, Davos (CH)

Correspondence Address: PENNIE & EDMONDS LLP, 1667 K STREET NW, SUITE 1000, WASHINGTON, DC, 20006

(21) Appl. No.: 10/162,658

(22) Filed: Jun. 6, 2002

Related U.S. Application Data
(63) Continuation-in-part of application No. PCT/CH99/00582, filed on Dec. 6, 1999.

(51) Int. Cl. A61B 17/80

(52) U.S. Cl. 606/69

(57) ABSTRACT

The invention relates to a resorbable bone plate used for adherence to soft tissue. It comprises an upper surface, a curved lower surface for contacting a bone, a plurality of conical through holes connecting the upper surface with the lower surface for receiving bone screws, a central longitudinal axis and consists of a resorbable material. The curved lower surface has a radius of curvature R which is smaller than 10 mm. The ratio F/d of the total area F of the upper surface including the surface area of the through holes to the thickness d of the plate is in the range of 190 to 270 mm. The central part of the longitudinal plate has a generally constant width B and the width of the free ends of the longitudinal plate narrows to a value b<0.3 B. The resorbable bone plate overcomes the problems of adherence of soft-tissue. Its shape and function is optimized given the limitations of the properties of these materials.

### CROSS-REFERENCE TO RELATED APPLICATIONS

This application is a continuation of the U.S. national stage designation of copending International Patent Application PCT/CH99/00582, filed Dec. 6, 1999, the entire content of which is expressly incorporated herein by reference thereto.

### FIELD OF THE INVENTION

The present invention relates generally to a resorbable bone plate.

### BACKGROUND OF THE INVENTION

Bone plates made of titanium have shown that this material has such a bio-compatibility that soft-tissue is adhering thereto which can be in certain situations an undesirable property for bone plates. On the other hand bone plates made of bioresorbable materials usually have insufficient mechanical properties (e.g., stiffness).

The invention as claimed aims at overcoming the problems of adherence of soft-tissue and to optimize shape and function of a resorbable plate given the limitations of the properties of these materials.

### SUMMARY OF THE INVENTION

The invention relates to a resorbable bone plate used for adherence to soft tissue. It comprises an upper surface, a curved lower surface for contacting a bone, a plurality of conical through holes connecting the upper surface with the lower surface for receiving bone screws, a central longitudinal axis and consists of a resorbable material. The curved lower surface has a radius of curvature R which is smaller than 10 mm. The ratio F/d of the total area F of the upper surface including the surface area of the through holes to the thickness d of the plate is in the range of 190 to 270 mm. The central part of the longitudinal plate has a generally constant width B and the width of the free ends of the longitudinal plate narrows to a value b<0.3 B.

Resorbable materials to be used for the device according to the invention can be resorbable polymers like highly purified polyhydroxyacids, polysaccharides, polyamines, polyaminoacids, polyorthoesters, polyanhydrides, polyamidoesters, polydioxanone, polyesteramides, copolyoxalates, polycarbonates or poly(glutamic-co-leucine). Preferably polylactides are used or their combinations with polyhydroxybutyrates or polyhydroxyvalerates and/or resorbable glasses.

Other useful polyhydroxyacids comprise polycaprolactone, poly(L-lactide), poly(DL-lactide), polyglycolide, poly(DL-lactide-co-glycolide), poly(DL-lactide-co-caprolactone).

Preferably the lower surface of the bone plate has a radius of curvature R which is smaller than 9 mm. The axis of the hollow cylinder corresponding to the curved plate is running parallel to the central longitudinal axis of the plate.

The ratio F/d of the total area F of the upper surface (including the surface area of the through holes) to the thickness d of the plate is in the range of 210 to 240 mm.

Preferably the ratio F/f of the total area F of the upper surface (including the surface area of the through holes) to the true surface f of the upper surface (excluding the surface area of the through holes) is in the range of 1.10-1.25.

The width of the free ends of the longitudinal plate is narrowing to value b being preferably smaller than 0.15 B.

The upper surface of the bone plate has preferably a roughness of 0.5 μm to 5.0 μm, typically of 0.6 μm to 2.5 μm in order to avoid the adherence of soft tissue after implantation. In any case it is preferred that the upper surface is smoother than the lower surface.

Preferably the bone plate is provided with a torsion around its central longitudinal axis which allows application to the required bone profile. The degree of torsion of the bone plate is preferably between ±1° to ±20° over the total length.

The length 1 of the central part of the longitudinal plate corresponds preferably to 73-82% of the total length L of the plate.

In order to achieve better angular stability selected or all of the conical through holes may be provided with a thread. However, it is also possible to use through holes with a smooth inner surface.

The through holes have preferably a cone angle of 15° to 25°, typically of 18° to 22°.

The bone plate is provided preferably with n≧4 holes (more preferably with n ≧6 holes), whereby the two most centrally located holes have preferably a distance which is at least 150%, preferably 180% of the distance x between the other through holes. When the central part of the plate is reinforced by omitting the most central hole (or holes) maximum rigidity of the cross section is obtained which is important for the bridging of bone defects.

### BRIEF DESCRIPTION OF THE DRAWINGS

Preferred features of the present invention are disclosed in the accompanying drawings, wherein similar reference characters denote similar elements throughout the several views, and wherein:

FIG. 1 is a view in elevation of the bone plate according to the invention; and

FIG. 2 is a cross-sectional view taken along line A-A of FIG. 1;

### DETAILED DESCRIPTION OF THE PREFERRED EMBODIMENTS

For convenience, the same or equivalent elements in the various embodiments of the invention illustrated in the drawings have been identified with the same reference numerals. Further, in the description that follows, any reference to either orientation or direction is intended primarily for the convenience of description and is not intended in any way to limit the scope of the present invention thereto.

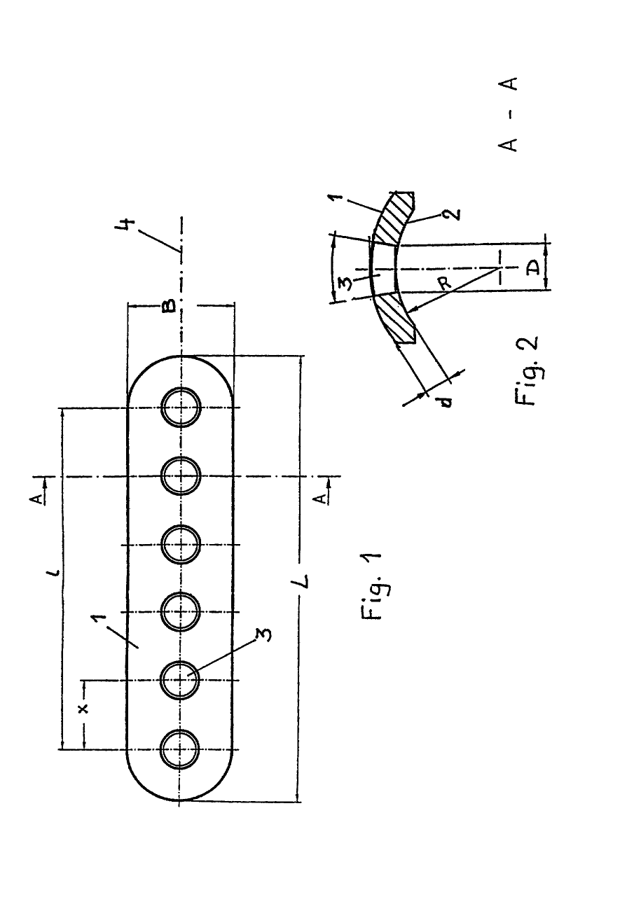

FIG. 1 shows a bone plate of longitudinal shape having an upper curved surface 1, a curved lower surface 2 for contacting a long bone, e.g. a metacarpal bone, six conical through holes 3 connecting the upper surface 1 with the lower surface 2 for receiving bone screws and a central longitudinal axis 4.

The through holes 3 have a cone angle of 200° and a minimum diameter D of 2.8 mm located at the lower side 2.

The lower surface 2 has a radius of curvature R of 7 mm. The plate consists of polylactide copolymer with an enhanced degradation rate.

The ratio F/d of the total area F of the upper surface 1 (including the surface area of the through holes 3) to the thickness d of the plate has the value of 223 mm. The ratio F/f of the total area F of the upper surface 1 including the surface area of the through holes 3 to the true surface f of the upper surface 1 excluding the surface area of the through holes 3 has the value of 1.17.

The central part of the longitudinal plate (over the length 1 in FIG. 1) has a constant width B whereas the width of the free ends of the longitudinal plate is narrowing gradually to value 0 in the form of a hemi-circle. The length l of the central part of the longitudinal plate corresponds to 77% of the total length L of the plate.

While various descriptions of the present invention are described above, it should be understood that the various features can be used singularly or in any combination thereof. Therefore, this invention is not to be limited to only the specifically preferred embodiments depicted herein. Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A longitudinal bone plate comprising:
a curved lower surface having a radius of curvature smaller than 10 mm;
a ratio F/d of the total area F of an upper surface, including a surface area defining a plurality of through holes, to a thickness d of the plate is in a range of 190 to 270 mm; and
a central part of the longitudinal bone plate with a generally constant width B, wherein the width of the free ends of the longitudinal plate narrows to a value b less than 0.3 B.

2. The bone plate of claim 1, wherein the radius of curvature is smaller than 9 mm.

3. The bone plate of claim 1, wherein the ratio F/d is in the range of 210 to 240 mm.

4. The bone plate of claim 1, wherein a ratio F/f of the total area F of the upper surface, including the surface area of the through holes to a true surface f of the upper surface, excluding the surface area of the through holes, is in a range of 1.10-1.25.

5. The bone plate of claim 1, wherein the width of the free ends of the longitudinal plate is narrowing to a value b less than 0.15 B.

6. The bone plate of claim 1, wherein the upper surface has a roughness of 0.5 μm to 5.0.

7. The bone plate of claim 1, wherein the upper surface is smoother than the lower surface.

8. The bone plate of claim 1, wherein the plate is provided with a torsion around its central longitudinal axis.

9. The bone plate of claim 8, wherein the length of the central part of the longitudinal plate corresponds to 73-82% of a total length L of the plate.

10. The bone plate of claim 1, wherein the through holes have a cone angle of 15° to 25°.

11. The bone plate of claim 10, wherein the through holes are provided with a thread.

12. The bone plate of claim 10, wherein the through holes have a smooth inner surface.

13. The bone plate of claim 1, wherein the plate defines at least 4 holes and the two most centrally located holes are spaced a distance which is at least 120% of the distance between the other holes.
